(19) United States
(12) Patent Application Publication
(10) Pub. No.: US 2024/0352076 A1
(43) Pub. Date: Oct. 24, 2024

(54) SPECIFIC BACULOVIRUS MAJOR ENVELOPE GLYCOPROTEIN GP64 BINDING PROTEINS

(71) Applicant: Navigo Proteins GmbH, Halle/Saale (DE)

(72) Inventors: Erik Fiedler, Halle/Saale (DE); Anja Katzschmann, Halle/Saale (DE); Heike Boecker, Halle/Saale (DE); Maren Meysing, Halle/Saale (DE); Jonathan Lotze, Halle/Saale (DE); Hanna Bobolowski, Halle/Saale (DE)

(73) Assignee: Navigo Proteins GmbH, Halle/Saale (DE)

(21) Appl. No.: 18/629,324

(22) PCT Filed: Oct. 7, 2022
(86) PCT No.: PCT/EP2022/077921

§ 371 Date: Apr. 8, 2024

(30) Foreign Application Priority Data
Oct. 8, 2021 (EP) 21201661.2

(51) Int. Cl. C07K 14/005; C07K 1/22; C07K 14/245; G01N 33/569; G01N 33/68

(52) U.S. Cl. CPC C07K 14/005; C07K 1/22; C07K 14/245; G01N 33/56983; G01N 33/68; C12N 2710/14151; G01N 2333/01

(57) ABSTRACT

gp64 is the major envelope glycoprotein of baculoviruses. The present invention relates to novel proteins that specifically bind to the baculovirus envelope protein gp64. The novel proteins of the present invention are advanced and powerful tools because they allow precise capturing of gp64 in affinity chromatography. The gp64 binding proteins are particularly useful tools within the process of protein production (e.g. vaccine production) to provide for gp64 free samples. Further, the binding protein for gp64 are useful for methods to analyze the presence of gp64.

### TECHNICAL FIELD

gp64 is the major envelope glycoprotein of baculoviruses. The present invention relates to novel proteins that specifically bind to the baculovirus envelope protein gp64. The novel proteins of the present invention are advanced and powerful tools because they allow precise capturing of gp64 in affinity chromatography. The gp64 binding proteins are particularly useful tools within the process of protein production (e.g. vaccine production) to provide for gp64 free samples. Further, the binding protein for gp64 are useful for methods to analyze the presence of gp64.

### BACKGROUND OF THE INVENTION

For high level expression of biologically active proteins, an insect cell expression vector system wherein cells are transduced by a baculovirus is a well-established tool (also referred to as baculovirus-insect cell expression system or baculovirus expression vector system). Upon replication of baculovirus expression vectors in cultured insect cells or larvae, high yields of exogenous proteins are obtained. This baculovirus-insect cell expression system is an approved system to express viral proteins, for example, for the purpose of production of vaccines. However, a major contamination in such preparation is glycoprotein 64 (also referred to as gp64). gp64 is a major envelope protein, for example of the budded baculovirus Autographa californica nucleopolyhedro virus (AcNPV), required for viral entry and for efficient budding. However, in mammals, gp64 is immunogenic and may trigger a specific immune response. Needless to say that the contamination is highly undesirable in vaccine preparations. Vaccines that are used in medicine must be provided in highly purified form without any contaminants.

However, current processes within vaccine production involve complicated procedures of several purification steps to remove the contaminant protein gp64.

There is a strong need in the art to provide methods for a more simplified and efficient purification of vaccines by removing (capturing) the major contaminant gp64 from the preparation. The present invention meets this need by providing novel binding proteins for gp64. These novel binding proteins are particularly advantageous because they allow a precise capturing of gp64, in particular via affinity chromatography. This will enable successful purification of vaccines that were produced via a baculovirus-insect cell expression system.

The above overview does not necessarily describe all problems solved by the present invention.

### SUMMARY OF THE INVENTION

The present disclosure provides the following items 1 to 15, without being specifically limited thereto:
1. A binding protein for the baculoviral envelope protein gp64 comprising an amino acid sequence with at least 90% sequence identity to anyone selected from the group of SEQ ID NOs: 1-3 wherein the binding protein has a binding affinity of less than 100 nM for gp64.
2. The binding protein for gp64 according to item 1, wherein 2, 3, 4, 5, or 6 binding proteins are linked to each other.
3. The binding protein for gp64 according to items 1-2 wherein the binding protein is fused to at least one non-gp64 binding protein.
4. The binding protein for gp64 according to items 1-3 wherein the binding protein is fused to one or two non-gp64 binding protein(s) with at least 89% identity to SEQ ID NO: 6.
5. A fusion protein comprising the binding protein according to claim 1 and one or two non-gp64 binding protein(s) with at least 89% identity to SEQ ID NO: 6.
6. The fusion protein according to claim 5 wherein said fusion protein comprises an attachment site for site-specific coupling to a solid support.
7. The binding protein for gp64 according to any one of items 1-4 or the fusion protein of items 5-6 for use in technical applications such as affinity chromatography.
8. An affinity separation matrix comprising a binding protein for gp64 according to any one of items 1-4 or the fusion protein of items 5-6.
9. Use of the binding protein for gp64 according to any one of items 1-4, or the fusion protein of items 5-6 or the affinity separation matrix according to item 8 for affinity chromatography.
10. A method of affinity capturing of gp64, the method comprising:
   (i) providing a liquid that contains a gp64;
   (ii) providing an affinity separation matrix comprising at least one binding protein for gp64 according to any one of items 1-4 or the fusion protein of items 5-6 coupled to said affinity separation matrix;
   (iii) contacting said affinity separation matrix with the liquid under conditions that permit binding of the at least one binding protein for gp64 according to any one of items 1-4 or the fusion protein of items 5-6; and
   (iv) eluting said gp64 from said affinity purification matrix.
11. Use of the binding protein for gp64 according to items 1-4 or the fusion protein of items 5-6 or the affinity matrix of item 8 in methods to determine the presence of gp64.
12. A method of analyzing the presence of gp64 in liquid samples, the method comprising the following steps:
   (i) providing a liquid that contains gp64,
   (ii) providing the binding protein for gp64 according to items 1-4 or the fusion protein of items 5-6,
   (iii) contacting the liquid of (i) with the binding protein for gp64 according to items 1-4 or the fusion protein of items 5-6 under conditions that permit binding of the binding protein to the gp64,
   (iv) isolating the complex of gp64 and binding protein, and
   (v) determining the amount of the binding protein for gp64 in the liquid of (i).
13. Use of the binding protein for gp64 according to items 1-4 or the fusion protein of items 5-6 or the affinity matrix of claim 8 in methods of production of a vaccine.
14. A method of production of a vaccine protein, the method comprising the following steps:
   (i) providing a sample that contains a vaccine protein expressed in an insect cell expression system wherein the cells were transduced by a baculovirus;
   (ii) providing at least one binding protein for baculoviral gp64 according to any one of items 1-4 or the fusion protein of items 5-6;
   (iii) contacting said binding protein for baculoviral gp64 with the sample under conditions that permit binding of the binding protein for gp64 according to any one of items 1-4 or the fusion protein of items 5-6;
   (iv) separating the complex of the gp64 bound to said binding protein or fusion protein from said vaccine protein; and
   (v) isolating the vaccine protein.
15. The method of production of a vaccine protein according to claim 14, wherein in step b, the binding protein according to any one of items 1-4 or the fusion protein of items 5-6 is coupled to said affinity separation matrix.

This summary of the invention is not limiting, and other aspects and embodiments of the invention will become evident from the following description, examples and drawings.

### BRIEF DESCRIPTION OF THE FIGURES

FIG. 1: Amino acid sequences of binding proteins for gp64 (SEQ ID NOs: 1-3).

FIG. 2: Purification of 218433 (fusion protein comprising SEQ ID NO: 2) via HiTrap SP Sepharose HP and Tricorn 10/300 S75 increase. FIG. 2A shows the size exclusion chromatography (SEC) as final polishing step during purification of 218433. Solid black line indicates UV 280 nm signal. Conductivity is shown as dashed gray line; FIG. 2B shows SDS-PAGE of analyzed fractions from size exclusion chromatography. Purified 218433 shows a MW band between 20-23 kDa. Lane 1: protein marker, lane 2: first peak at retention time 10-11 min, lane 3-6: main peak at retention time 11-14 min.

FIG. 3: SPR of gp64 binding proteins immobilized via c-terminal cysteine residue using PDEA coupling on high amine chip (Bruker). FIG. 3A shows SPR of 218433 (fusion protein comprising gp64 binding protein SEQ ID NO: 2) with an affinity of 218433 to gp64 of 54 nM.

FIG. 3B shows SPR of 218441 (fusion protein comprising gp64 binding protein SEQ ID NO: 3) with an affinity of 218441 to gp64 of 31 nM.

FIG. 4: Purification of gp64 via affinity ligand immobilized to Praesto 85. FIG. 4A shows a SDS-PAGE of analyzed fractions of an affinity chromatography run performed with 218433 coupled to Praesto 85 resin. Purified gp64 from cell culture supernatant is detected between MW band 150-200 kDa under non-reduced conditions. No significant impurities were detectable. Lane 1: protein marker, lane 2-6: flowthrough fractions, lane 7-11: elution fractions. FIG. 4B shows SDS-PAGE of analyzed fractions of an affinity chromatography run performed with 218441 coupled to Praesto 85 resin. Purified gp64 from cell culture supernatant is between MW band 150-200 kDa under non-reduced conditions and between 60-70 kDa under reduced conditions. No significant impurities were detectable. Lane 1: protein marker, lane 2: cell culture supernatant, lane 3-9: flowthrough fractions, lane 10: elution fraction, lane 11: elution fraction under reduced conditions.

FIG. 5: Eluted protein identified by Western Blot as gp64 (Praesto 85_218433). The figure shows a western blot of analyzed fractions of an affinity chromatography run performed with 218433 coupled to Praesto 85 resin. Samples were applied under reducing conditions and identified by Acm-NPV gp64 polyclonal antibody (primary antibody 1:20.000 dilution) and goat-anto-rabbit IgG-HRP (secondary antibody 1:20.000 dilution). Purified target gp64 from cell culture supernatant is between MW band 50-75 kDa under reduced conditions. Lane 1: protein marker, lane 2: cell culture supernatant, lane 3-10: flowthrough fractions, lane 11-12: elution fraction.

### DETAILED DESCRIPTION OF THE INVENTION

The present invention provides novel proteins having specific binding affinity for gp64. The novel proteins of the present invention are particularly advantageous because as affinity ligands for gp64, they allow precise purification (capturing) of gp64, for example in affinity chromatography. Any polypeptide selected from the group of SEQ ID NO: 1, SEQ ID NO: 2, and SEQ ID NO: 3, or an amino acid sequence with at least 90% identity to any one of SEQ ID NO: 1, SEQ ID NO: 2, and SEQ ID NO: 3, respectively, bind to gp64 and are suitable for example for the removal of the contaminant gp64 from a protein preparation, for example, a vaccine preparation.

Before the present invention is described in more detail below it is to be understood that this invention is not limited to the particular methodology, protocols and reagents described herein as these may vary. It is also to be understood that the terminology used herein is for the purpose of describing particular aspects and embodiments only and is not intended to limit the scope of the present invention, which is reflected by the appended items. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new specific binding molecules for gp64 for use in technical applications, for example for use as affinity ligands in affinity chromatography.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the items, which follow, unless the context requires otherwise, the word “comprise”, and variants such as “comprises” and “comprising”, will be understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, UniProt Accession Number, etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, forms part of the disclosure content of the present specification.

### General Definitions of Important Terms Used in the Application

The term “gp64” relates to baculovirus envelope glycoprotein gp64 and refers to an amino acid 25 sequence as shown in UniProtKB P17501. The term “gp64” comprises all polypeptides which show an amino acid sequence identity of at least 70%, 80%, 85%, 90%, 95%, 96% or 97% or more, or 100% to UniProtKB P17501.

The terms “binding protein for gp64” or “gp64 binding protein” or “affinity ligand”” may be used interchangeably herein and describe a protein that is capable to bind to gp64. As described herein, a binding protein for gp64 refers to a protein with detectable interaction with gp64, as determined by suitable methods such as for example SPR analysis or BLI or other appropriate technology known to someone skilled in the art.

The term “non-gp64 binding protein” refers to a protein with no detectable interaction with gp64, as determined by suitable methods such as for example SPR analysis or BLI or other appropriate technology known to someone skilled in the art.

The term “binding affinity” refers to the ability of a polypeptide of the invention to bind to gp64. Binding affinity is typically measured and reported by the equilibrium dissociation constant (K_{D}) which is used to evaluate and rank the strength of bimolecular interactions. The binding affinity and dissociation constants can be measured quantitatively. Methods for determining binding affinities are well known to the skilled person and can be selected, for instance, from the following methods that are well established in the art: surface plasmon resonance (SPR), Bio-layer interferometry (BLI), enzyme-linked immunosorbent assay (ELISA), kinetic exclusion analysis (KinExA assay), flow cytometry, fluorescence spectroscopy techniques, isothermal titration calorimetry (ITC), analytical ultracentrifugation, radioimmunoassay (RIA or IRMA), and enhanced chemiluminescence (ECL). Typically, the dissociation constant K_{D} is determined at temperatures in the range of 20° C. and 30° C. If not specifically indicated otherwise, K_{D} values recited herein are determined at 25° C. by SPR. In various embodiments of the present invention, the binding affinity for gp64 may be determined by the Sierra SPR-32 system (Bruker).

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide, for example, to the polypeptide of SEQ ID NO: 2. Methods for sequence alignment are well known in the art. For example, for determining the extent of an amino acid sequence identity of an arbitrary polypeptide relative to the amino acid sequence of, for example, SEQ ID NO: 2, the SIM Local similarity program is preferably employed (Xiaoquin Huang and Webb Miller (1991), Advances in Applied Mathematics, vol. 12: 337-357), that is freely available. For multiple alignment analysis, ClustalW is preferably used (Thompson et al. (1994) Nucleic Acids Res., 22(22): 4673-4680).

The terms “protein” and “polypeptide” refer to any chain of two or more amino acids linked by peptide bonds and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide like, e.g., glycosylation, which are well known in the art.

The term “alkaline stability” or “caustic stability” refers to the ability of the binding protein for gp64 to withstand alkaline conditions without significantly losing the ability to bind gp64. The skilled person in this field can easily test alkaline stability by incubating a binding protein for gp64 with sodium hydroxide solutions, e.g., as described in the Examples, and subsequent testing of the binding affinity to gp64 by routine experiments known to someone skilled in the art, for example, by chromatographic approaches.

The term “chromatography” refers to separation technologies which employ a mobile phase and a stationary phase to separate one type of molecules (e.g., gp64) from other molecules (e.g., a vaccine protein) in the sample. The liquid mobile phase contains a mixture of molecules and transports these across or through a stationary phase (such as a solid matrix). Due to the differential interaction of the different molecules in the mobile phase with the stationary phase, molecules in the mobile phase can be separated.

The term “affinity chromatography” refers to a specific mode of chromatography in which a ligand (i.e. a binding protein for gp64) coupled to a stationary phase interacts with a molecule (i.e. gp64) in the mobile phase (the liquid sample), i.e. the ligand has a specific binding affinity for the molecule to be captured. As understood in the context of the invention, affinity chromatography involves the addition of a (liquid) sample containing gp64 to a stationary phase which comprises a chromatography ligand, such as a binding protein for gp64. The terms “solid support” or “solid matrix” are used interchangeably for the stationary phase.

The terms “affinity matrix” or “affinity purification matrix”, as used interchangeably herein, refer to a matrix, e.g., a chromatographic matrix, onto which an affinity ligand (e.g., a binding protein for gp64) is attached. The attached affinity ligand (e.g., binding protein for gp64) is capable of specific binding to a molecule of interest (e.g., gp64) which is to be purified or removed (captured) from a (liquid) sample.

The term “affinity purification” or “affinity capturing” as used herein refers to a method of purifying (capturing) gp64 from a liquid sample by binding gp64 to a ligand for gp64 that is immobilized to a matrix. Thereby, gp64 is removed (captured) from the liquid.

The term “vaccine protein” or “protein vaccine” as used herein refers to parts or proteins or fragments or subunits of a protein of a pathogen, such as a virus or bacterium.

### Detailed Description of the Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

The novel binding protein for gp64 exhibit a specific binding affinity for the gp64. The binding protein for gp64 comprises the amino acid sequence selected from the group of SEQ ID NO: 1, SEQ ID NO: 2, SEQ ID NO: 3, or an amino acid with at least 90% sequence identity to any one of SEQ ID NO: 1, SEQ ID NO: 2, SEQ ID NO: 3. In some embodiments, the binding protein for gp64 comprises the amino acid sequence of SEQ ID NO: 1. SEQ ID NO: 1 comprises the amino acid sequence of AKFDEAQSAADSEILHLPNLTEXQRXXFRXXLXXXPSVSXXXLXXAQXXNDXQAPK, wherein X may be any one of A, R, N, D, C, Q, E, G, H, I, L, K, M, F, P, S, T, W, Y, or V and is preferably any one of H or Y at position 23, S or N at position 26, W or I at position 27, W or Y at position 30, S or M at position 33, D or R at position 35, K or L at position 40, E or Q at position 41, T or V at position 42, E or R at position 44, Q or W at position 45, and R or Q at position 52, or any combination thereof. In some embodiments, the binding protein for gp64 comprises or an amino acid with at least 90% sequence identity to SEQ ID NO: 1.

In some embodiments, the binding protein for gp64 comprises the amino acid sequence of SEQ ID NO: 2. In some embodiments, the binding protein for gp64 comprises an amino acid with at least 90% sequence identity to SEQ ID NO: 2.

In some embodiments, the binding protein for gp64 comprises the amino acid sequence of SEQ ID NO: 3. In some embodiments, the binding protein for gp64 comprises an amino acid with at least 90% sequence identity to SEQ ID NO: 3.

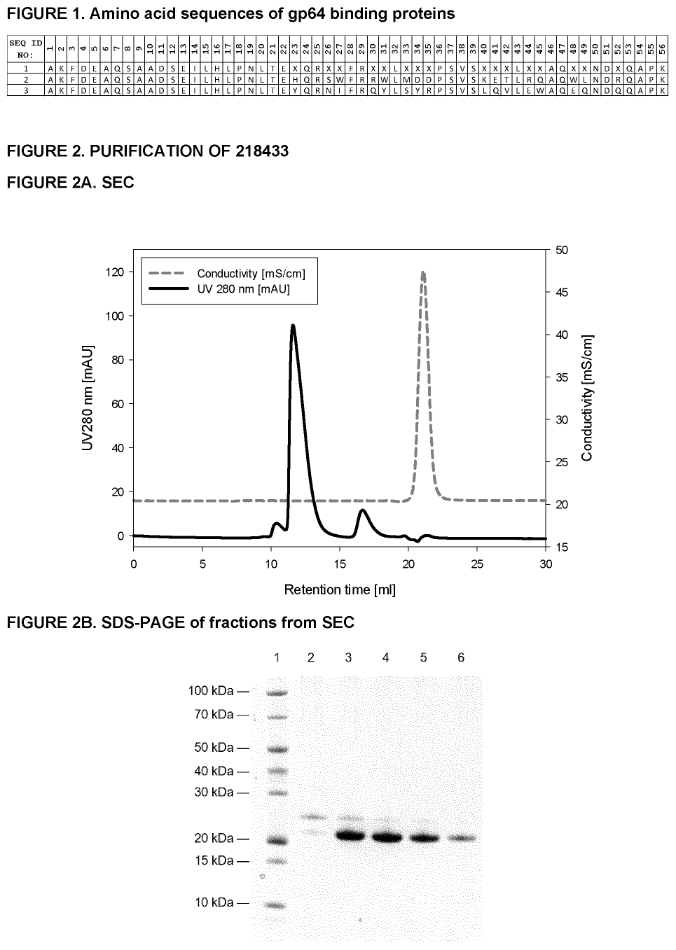

In some embodiments, a binding protein for gp64 is comprising at least one amino acid sequence as shown in FIG. 1.

In some embodiments, the binding protein for gp64 has at least 90%, 91%, 92%, 93%, 94%, 95%, 96%, 97%, 98%, 99%, or 100% sequence identity to any one of the amino sequences of SEQ ID NOs: 1-3. In some embodiments, amino acids 1-22, 24, 25, 28, 29, 36-39, 43, 46, 47, 50, 51, and 53-56 are identical in gp64 binding proteins as shown in the amino acid sequences of SEQ ID NOs: 1-3. In other embodiments, amino acids 1-22, 24, 25, 28, 36-39, 43, 46, 47, 50, 51, and 53-56 are identical in gp64 binding proteins as shown in the amino acid sequences of SEQ ID NOs: 1-3.

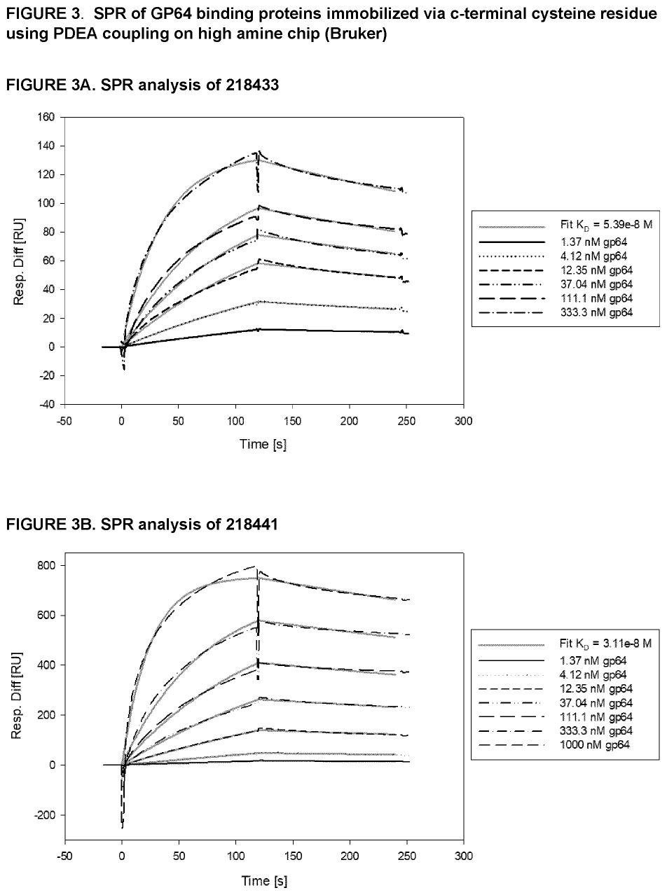

One advantage of the herein disclosed binding protein for gp64 is the important functional characteristic that it binds specifically to gp64. Needless to point out, that this is of particular advantage in capturing of the contaminant gp64 in the process of protein purification, for example in the process of vaccine production (e.g. vaccine purification). The binding protein for gp64 is functionally characterized by a binding affinity of less than 100 nM for gp64, as shown in the FIG. 3 and in Example 3. In some embodiments, the binding protein for gp64 is functionally characterized by a binding affinity of less than 60 nM for gp64, as shown in the FIG. 3 and in Example 3.

Multimers. In one embodiment of the invention, the binding protein for gp64 comprises 1, 2, 3, 4, 5, or 6 binding protein(s) linked to each other. In one embodiment of the invention, the binding protein for gp64 comprises one gp64 binding protein or two gp64 binding proteins linked to each other. Multimers of the binding protein are generated artificially, generally by recombinant DNA technology well-known to a skilled person. In some embodiments, the multimer is a homo-multimer, e.g. the amino acid sequences of binding protein for gp64 are identical. In other embodiments, the multimer is a hetero-multimer, e.g. the amino acid sequences of the binding protein for gp64 are different.

Fusion proteins. In some embodiments, the binding protein for gp64 as described above is fused to one further polypeptide distinct from the polypeptide as disclosed. In various embodiments, the further polypeptide distinct from the binding protein for gp64 as disclosed herein might be a non-gp64 binding protein. In some embodiments, the further non-gp64 binding polypeptide is a non-Immunoglobulin (Ig)-binding protein, for example but not limited to, a protein that does not bind to Ig. In some embodiments, a common structural feature of the non-gp64 binding proteins is that they have a triple helical Protein A-like structure comparable to the structure of the gp64 binding proteins as disclosed herein.

In some embodiments, a non-gp64 binding protein has an amino acid sequence identity of at least 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 6. In some embodiments, a non-gp64 binding protein has an amino acid sequence identity of at least 89% to SEQ ID NO: 6.

Some embodiments encompass fusion proteins comprising a binding protein for gp64 as disclosed herein and at least one non-gp64 binding polypeptide.

Some embodiments encompass fusion proteins comprising a binding protein for gp64 as disclosed herein and one or two or more non-gp64 binding polypeptide(s).

In some embodiments, fusion proteins comprising at least one binding protein for gp64 of anyone selected from SEQ ID NOs: 1-3 and at least one non-gp64 binding protein.

In some embodiments, fusion proteins comprising at least one binding protein for gp64 of anyone selected from SEQ ID NOs: 1-3 and at least one non-gp64 binding protein of at least 89% identity to SEQ ID NO: 6.

In some embodiments, the fusion protein is comprising a binding protein for gp64 wherein C-terminus of the binding protein for gp64 is fused to the N-terminus of a non-gp64 binding protein.

In some embodiments, the fusion protein is comprising at least one binding protein for gp64 wherein C-terminus of the binding protein for gp64 is fused to the N-terminus of a dimer of a non-gp64 binding protein with at least 89% or at least 90% identity to SEQ ID NO: 6.

In one embodiment, a fusion protein comprises SEQ ID NO: 2 fused to two non-gp64 binding proteins; the amino acid sequence of the fusion protein is provided in SEQ ID NO: 4.

In one embodiment, a fusion protein comprises SEQ ID NO: 3 fused to two non-gp64 binding proteins; the amino acid sequence of the fusion protein is provided in SEQ ID NO: 5.

In one embodiment, a fusion protein comprises a gp64 binding protein with at least 98% amino acid identity to SEQ ID NO: 2 fused to two non-gp64 binding proteins; the amino acid sequence of the fusion protein is provided in SEQ ID NO: 8, SEQ ID NO: 9, and SEQ ID NO: 10.

In some embodiments said fusion protein comprises an attachment site for site-specific coupling to a solid support, as further described below.

Molecules for purification or detection. In some embodiments, the binding protein for gp64 may also comprise additional amino acid residues at the N- and/or C-terminal end, such as for example an additional sequence at the N- and/or C-terminal end. Additional sequences may include for example sequences introduced e.g. for purification or detection. Typical examples for such sequences include, without being limiting, Strep-tags, oligohistidine-tags, glutathione S-transferase, maltose-binding protein, inteins, intein fragments, or the albumin-binding domain of protein G, or others. In one embodiment, additional amino acid sequences include one or more peptide sequences that confer an affinity to certain chromatography column materials. The binding protein for gp64 or a fusion protein comprising the binding protein for gp64 may include specific attachment sites for the attachment to solid supports, preferably at the C-terminal end, such as cysteine or lysine.

Use of the novel binding protein for gp64 in technical applications. Also provided herein is the use of any novel binding protein for gp64 as disclosed herein, including fusion proteins, in technical applications, preferably for use in affinity purification.

Affinity purification (capturing) of gp64. As described herein, affinity chromatography (also referred to as affinity purification) makes use of specific binding interactions between molecules.

Methods for immobilization of protein and methods for affinity chromatography are well-known in the field of protein purification and can be easily performed by a skilled person in this field using standard techniques and equipment.

Some embodiments refer to a method of affinity capturing (purification) of gp64, the method comprising: (i) providing a liquid that contains a gp64 (such as gp64 as contaminant in vaccine protein preparations); (ii) providing an affinity separation matrix comprising at least one binding protein for gp64 as described above or the fusion protein as described above coupled to said affinity separation matrix; (iii) contacting said affinity separation matrix with the liquid under conditions that permit binding of the at least one binding protein for gp64 as described above or the fusion protein as described above; and (iv) eluting said gp64 from said affinity purification matrix.

In various embodiments, the method of affinity purification may further comprise one or more washing steps. Affinity purification matrices suitable for the disclosed uses and methods are known to a person skilled in the art.

Conjugation to a solid support. In various aspects and/or embodiments of the present invention, the novel gp64 binding proteins disclosed herein including novel gp64 binding proteins generated or obtained by any of the methods as described above are conjugated to a solid support. In some embodiments, the gp64 binding protein comprises an attachment site for site-specific covalent coupling of the gp64 binding protein to a solid support. Specific attachment sites comprise without being limited thereto, natural amino acids, such as cysteine or lysine, which enable specific chemical reactions with a reactive group of the solid phase, or a linker between the solid phase and the protein.

Affinity purification matrix. In another embodiment, an affinity purification matrix is provided comprising a binding protein for gp64, or a fusion protein comprising the binding protein for gp64. In preferred embodiments, the affinity purification matrix is a solid support. The affinity purification matrix comprises at least one binding protein for gp64 or a fusion protein comprising the binding protein for gp64 as described herein. Accordingly, a novel binding protein for gp64 disclosed herein or a fusion protein comprising the binding protein for gp64 is encompassed for use in the purification (capturing) of gp64 by an affinity purification matrix.

Solid support matrices for affinity chromatography are known in the art and include, e.g., without being limited thereto, agarose and stabilized derivatives of agarose, cellulose or derivatives of cellulose, controlled pore glass, monolith, silica, zirconium oxide, titanium oxide, or synthetic polymers, and hydrogels of various compositions and combinations of the above.

The formats for solid support matrices can be of any suitable well-known kind. Such solid support matrix for coupling a novel protein or polypeptide of the present invention might comprise, e.g., one of the following, without being limited thereto: columns, capillaries, particles, membranes, filters, monoliths, fibers, pads, gels, slides, plates, cassettes, or any other format commonly used in chromatography and known to someone skilled in the art.

In one embodiment, the matrix is comprised of substantially spherical beads, for example agarose beads (for example, a polysaccharide polymer material in crosslinked form also known as Sepharose). Matrices in particle form can be used as a packed bed or in a suspended form including expanded beds. In other embodiments of the invention, the solid support matrix is a membrane, for example a hydrogel membrane. In some embodiments, the affinity purification may involve a membrane as a matrix to which the binding protein for gp64 as described herein is covalently bound. The solid support can also be in the form of a membrane in a cartridge.

In some embodiments, the affinity purification involves a chromatography column containing a solid support matrix to which a novel protein of the present invention is covalently bound. The gp64 binding protein or fusion protein comprising the gp64 binding protein as described above may be attached to a suitable solid support matrix via conventional coupling techniques. Methods for immobilization of protein ligands to solid supports are well-known in the field of protein engineering and purification and can easily be performed by a skilled person in this field using standard techniques and equipment.

Use and methods to determine the presence of a gp64. In some embodiments refer to a use of the binding protein for gp64 as described above or the fusion protein as described above or the affinity matrix as described above in methods to determine the presence of gp64.

Some embodiments, the binding protein for gp64 as described herein or the fusion protein as described herein is used in methods to determine the presence of a gp64. Some embodiments relate to a method of analyzing the presence of gp64 in liquid samples, the method comprising the following steps: (i) providing a liquid that contains gp64, (ii) providing the binding protein or the fusion protein for gp64, (iii) contacting the liquid that contains gp64 with the binding protein or the fusion protein for gp64 as described herein under conditions that permit binding of the at least one binding protein for gp64 to gp64, (iv) isolating (eluting) the complex of a gp64 and the binding protein or the fusion protein for gp64, and (v) determining the amount of the binding protein for gp64 which indicates the amount of gp64 in the liquid of (i).

Method of quantification of a gp64. Further embodiments relate to a method of quantification of a gp64, the method comprising: (i) providing a liquid that contains gp64; (ii) providing a matrix to which the binding protein or the fusion protein for gp64 as described herein has been covalently coupled; (iii) contacting said affinity purification matrix with the liquid under conditions that permit binding of the at least one binding protein or fusion protein for gp64 to gp64; (iv) eluting said gp64; and (v) quantitating the amount of eluted gp64. Methods to determine the presence of gp64 in liquid samples might be quantitative or qualitative. Such methods are well known to the skilled person and can be selected, for instance but limited to, from the following methods that are well established in the art: enzyme-linked immunosorbent assay (ELISA), enzymatic reactions, surface plasmon resonance (SPR) or chromatography.

Some embodiments refer to a use of the binding protein for gp64 as described above or the fusion protein as described above or the affinity matrix as described above in methods of production of a vaccine, in particular in methods for producing a vaccine protein.

Method of vaccine protein production (by capturing the contaminant gp64). A vaccine protein may contain parts or proteins or fragments (subunits) of a protein of a pathogen, such as a virus or bacterium. Some embodiments refer to a method of production of a vaccine protein, the method comprising the following step, in particular to remove the gp64 contamination from the vaccine protein preparation:
(i) providing a sample that contains a vaccine protein expressed in an insect cell expression system wherein the cells were transduced by a baculovirus;
(ii) providing at least one binding protein for baculoviral gp64 as described above or the fusion protein as described above;
(iii) contacting said binding protein for baculoviral gp64 with the sample under conditions that permit binding of the binding protein for gp64 as described above or the fusion protein as described above;
(iv) separating the complex of the gp64 bound to said binding protein or fusion protein from said vaccine protein; and
(v) isolating the vaccine protein.

In some embodiments, in the method of production of a vaccine protein, in step (ii), the binding protein as described above or the fusion protein as described above is coupled to said affinity separation matrix.

In one embodiment, the method of production of a vaccine protein is comprising the following steps, in particular the following steps to remove the gp64 contamination from the vaccine protein preparation:
(i) providing a sample that contains a vaccine protein expressed in an insect cell expression system wherein the cells were transduced by a baculovirus;
(ii) providing an affinity separation matrix comprising at least one binding protein for baculoviral gp64 as described above or the fusion protein as described above coupled to said affinity separation matrix;
(iii) contacting said affinity separation matrix with the sample under conditions that permit binding of the immobilized binding protein for gp64 as described above or the fusion protein as described above;
(iv) collecting the flowthrough from the affinity separation matrix comprising said vaccine protein;
(v) eluting said contaminant gp64 from said affinity purification matrix; and
(vi) isolating the vaccine protein from the flowthrough.

Polynucleotides, vectors, host cells. One embodiment covers an isolated polynucleotide or nucleic acid molecule encoding a binding protein for gp64 as described herein. A further embodiment also encompasses proteins encoded by polynucleotides.

Further provided is a vector, in particular an expression vector, comprising the isolated polynucleotide or nucleic acid molecule for the gp64 binding protein as described herein, as well as a host cell comprising the isolated polynucleotide or the expression vector. For example, one or more polynucleotides, which encode the gp64 binding protein as disclosed herein may be expressed in a suitable host and the produced protein can be isolated. A vector means any molecule or entity (e.g., nucleic acid, plasmid, bacteriophage or virus) that can be used for transfer of protein-encoding information into a host cell. Suitable vectors that may be applied are known in the art.

Furthermore, an isolated cell comprising a polynucleotide or nucleic acid, or a vector as disclosed herein is provided. Suitable host cells include prokaryotes or eukaryotes, for example a bacterial host cell, a yeast host cell or a non-human host cell carrying a vector. Suitable bacterial expression host cells or systems are known in the art. Various mammalian or insect cell culture systems as known in the art can also be employed to express recombinant proteins.

Method of producing a protein of the invention. In a further embodiment, a method for the production of the binding protein or fusion protein for gp64 as described is provided, the method comprising the step(s): (i) culturing a (suitable) host cell under conditions suitable for the expression of the binding protein for gp64 or fusion protein so as to obtain said binding protein or fusion protein for gp64; and (ii) optionally isolating said binding protein or fusion protein for gp64. Suitable conditions for culturing a prokaryotic or eukaryotic host are well known to a person skilled in the art.

The binding protein for gp64 may be prepared by any conventional and well-known techniques such as plain organic synthetic strategies, solid phase-assisted synthesis techniques, or by commercially available automated synthesizers. They may also be prepared by conventional recombinant techniques, alone or in combination with conventional synthetic techniques.

In one embodiment, a method for the preparation of the binding protein or fusion protein for gp64 is provided, as detailed above, said method comprising the steps: (i) providing a nucleic acid molecule encoding the gp64 binding protein or fusion protein; (ii) introducing said nucleic acid molecule into an expression vector; (iii) introducing said expression vector into a host cell; (iv) culturing the host cell in a culture medium; (v) subjecting the host cell to culturing conditions suitable for expression thereby producing the gp64 binding protein or fusion protein; optionally (vi) isolating the polypeptide produced in step (v); and (vii) optionally conjugating the gp64 binding protein or fusion protein to a solid matrix as described above. In various embodiments of the present invention the production of the binding protein or fusion protein for gp64 is performed by cell-free in vitro transcription and translation.

The following Examples are provided for further illustration of the invention. The invention, however, is not limited thereto, and the following Examples merely show the practicability of the invention on the basis of the above description.

### EXAMPLES

The following Examples are provided for further illustration of the invention. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Selection and Screening

Libraries. Proprietary cDNA libraries based on stable Protein A-like variants (artificial mosaic proteins composed of fragments of Protein A domains and additional mutations) were synthesized in house by randomized oligonucleotides generated by synthetic trinucleotide phosphoramidites (ELLA Biotech) or synthesized extern by Geneart to achieve a well-balanced amino acid distribution with simultaneously exclusion of cysteine and other amino acid residues at randomized positions. For the following selection process by phage display, the corresponding cDNA library was amplified by PCR and ligated into a pCD33-OmpA phagemid. Aliquots of the ligation mixture were used for electroporation of E. coli SS320 (Lucigen) to produce and purify the phage library to store them as cryo-stocks.

For the following selection process by ribosome display these cDNA libraries were supplemented with a T7 promoter region at the 5′ end and a spacer region at the 3′ end, respectively. Unless otherwise indicated, established recombinant genetic methods were used.

Selection by phage display: Unless otherwise indicated, established recombinant genetic methods were used. Naive libraries were enriched against gp64-His (C-terminal His-tag) as ON-target using phage display as selection system. In each round a pre-selection step was performed using empty BSA-blocked magnetic Dynabeads M-270 Epoxy. The AIT-method was applied, which means that the ON-target protein was immobilized to magnetic Dynabeads M-270 Epoxy before each round started. E. coli SS320 (Lucigene) were used for infection with cryo phage libraries and E. coli ER2738 (Lucigene) for reamplification of phage pools after each round.

Amplification and purification of the phages were carried out using standard methods known to a skilled person. All three selection rounds were performed with the automated KingFisher-System (Thermo Fisher) to isolate and capture the desired phage-target complexes. Target concentration started at 120 nM (round 1) and declined each round down to 40 nM (round 3). Bound phages were eluted by trypsin and reamplified. The success of the selection was analyzed by phage-pool-ELISA in medium binding microtiter plate (Greiner Bio-One) coated with gp64-His (125 ng/well), S1-His (125 ng/well), hlgG₁-Fc (125 ng/well), BSA (125 ng/well) or Sigmablocker. Bound phages were detected using α-M13 HRP conjugated antibody (GE Healthcare).

Cloning of target binding phage pools into an expression vector. Selection pools showing specific binding to recombinant gp64-His in phage pool ELISA were amplified by PCR according to methods known in the art, cut with appropriate restriction nucleases and ligated into a derivative of the expression vector pET-28a (Merck, Germany) comprising an N-terminal GFP-His-tag followed by an enzymatic cleavage site and a C-terminal cysteine.

Various phage display selection pools resulted in specific signals for the respective ON-target gp64-His. Controls with S1-His, hlgG₁-Fc, BSA, or Sigmablocker showed no binding in the respective pools. Selected pools were sequenced and subcloned for high throughput screening.

Ribosome display: Proprietary cDNA libraries including the ribosome display regulatory elements were each transcribed into the corresponding RNA library followed by in vitro translation into a protein library. Those generated mRNA-ribosome-protein-ternary complexes were stable and thus suitable for selection. The ternary complexes were allowed to bind the target protein already immobilized on magnetic epoxy beads (Dynabeads M-270 Epoxy, Thermo Fisher Scientific). Target concentration started at 140 nM (round 1) and declined each round down to 50 nM (round 3). Selection pools of rounds 2 and 3 were amplified by PCR according to methods known in the art, cut with appropriate restriction nucleases and ligated into a derivative of the expression vector pET-28a (Merck, Germany) comprising an N-terminal GFP-His-tag followed by an enzymatic cleavage site and a C-terminal cysteine. To identify gp64-His specific selection pools, subcloned pools of rounds 2 and 3 were analyzed by pool ELISA. Therefore, subcloned pools were transformed in E. coli BL21(DE3), cultivated in 3 ml autoinduction media and the cells were harvested by three freeze/thaw cycles. Wells of a high binding black plate (Greiner Bio-One) were coated with recombinant gp64-His as ON-target (1.0 and 10 μg/ml respectively) and IgG₁-Fc and BSA (each 10 μg/ml) as OFF-targets. E. coli lysates were allowed to bind the targets followed by several washing steps. Bound variants were detected via fluorescence signal (ex 485 nm/em 535 nm). Several pools showed specific signals to recombinant gp64-His and were passed to high throughput screening.

In parallel, sequence analysis of single clones out of selected pools was performed, comprising sequence enrichment of up to 100%. Sequence enriched variants (15×) were selected and forwarded to μ-scale purification and BLI analysis.

Primary screening: 16 Selection pools (14×PhD, 2×RD) were proceeded to high-throughput screening vs. gp64-His (ON-target) and BSA (OFF-target). gp64-His and BSA [c=2.0 μg/ml] were immobilized on a 384-well high-binding plate and bound variants were detected by fluorescence signal (ex 485 nm/em 535 nm). 381 variants were selected for sequencing, μ-scale purification and BLI analysis. Hit criteria: soluble protein expression, signal of sample >5.000 and 5-fold larger than signal of OFF-target.

μ-scale purification and BLI analysis: 381 variants arrived from primary screening were selected for sequencing. Additionally with 15 sequence enriched variants arrived from RD selection a total of 396 variants were defined as hit for μ-scale purification and BLI analysis vs gp64-His using an Octet 8 channel system. For BLI measurements variants were immobilized via His-Tag on the surface of Ni-NTA sensors (ForteBio). Upon binding, variants were accumulated on the surface increasing the refractive index. This change in the refractive index was measured in real time and plotted as nm shift versus time. Affinity of captured variants vs gp64-His [c=250 nM] was determined and also plotted as nm shift versus time. 2 hit variants were selected for subcloning in a suitable coupling format and a production in lab scale.

### Example 2. Expression and Purification of Gp64 Binding Proteins

gp64 binding proteins were expressed in Escherichia coli BL21(DE3) using a pNP-016 vector system under regulation of a T7 promoter. Proteins were produced in soluble form after induction by lactose included in the medium (autoinduction medium). BL21 (DE3) competent cells were transformed with the expression plasmid, spread onto selective agar plates (kanamycin) and incubated over night at 37° C. Precultures were inoculated from single colony in 50 ml 2×YT medium supplemented with 50 μg/ml kanamycin and cultured for 7 h at 37° C. in shake flasks. For main cultures 350 mL autoinduction medium (modified H15 medium consisting of 2% glucose, 5% yeast extract, 0.89% glycerol, 0.76% lactose, 250 mM MOPS, 202 mM TRIS, 10 mM MgSO₄, pH 7.4, antifoam SE15) supplemented with 50 μg/ml kanamycin and trace elements (see Studier 2005) were inoculated to an OD₆₀₀ of 0.3 and incubated in 2.5 L Ultra Yield flasks at 37° C. in an orbital shaker. Recombinant protein expression was induced by metabolizing glucose and subsequently allowing lactose to enter the cells. Cells were grown over night for approximately 18 hours to reach a final OD₆₀₀ of about 40-50. Before the harvest, the OD₆₀₀ was measured, samples adjusted to 0.6/OD₆₀₀ were withdrawn, pelleted and frozen at −20° C. To collect biomass cells were centrifuged at 12000×g for 20 min at 22° C. Pellets were weighed (wet weight) and stored at −20° C. before processing.

The untagged proteins were purified by cation exchange chromatography and size exclusion. After cell disruption acetic acid was added to a final concentration of 100 mM followed by a pH-adjustment to pH 4.0 using hydrochloric acid. The initial capturing step was performed using SP Sepharose HP (Cytiva; binding buffer: 20 mM citric acid, 1 mM EDTA pH 3.5; elution buffer: 20 mM citric acid, 1 mM EDTA, 1 M NaCl pH 3.5) followed by a size exclusion chromatography (Superdex 75 26/600, Cytiva) in 20 mM citric acid, 150 mM NaCl, 1 mM EDTA, 5 mM TCEP pH 6.0 carried out on an AKTA avant system (Cytiva).

Following SDS-PAGE analysis positive fractions were pooled and the protein concentrations were determined by absorbance measurement at 280 nm using the molar absorbent coefficient. Further analysis included RP-HPLC and SE-HPLC. Reversed phase chromatography (RP-HPLC) has been performed using an Ultimate 3000 HPLC system (Thermo Fisher Scientific) and a PLRP-S (5 μm, 300 Å) column (Agilent). The resulting purity of fusion proteins 218433, d 218441, 220067, 220068, and 220069 was 100%. Analytic size exclusion chromatography (SE-HPLC) has been performed using an Ultimate 3000 HPLC system (Thermo Fisher Scientific) and a Superdex75 increase 5/150 GL (Cytiva). No aggregation or oligomers were obtained. Final yields for fusion proteins 218433 and 218441 were 0.82-0.84 mg per g wet biomass. Results of SEC and SDS-PAGE of fractions from SEC are shown in FIG. 2 for 218433.

### Example 3. Binding Analysis of Proteins by SPR

The purified proteins were immobilized on a High Capacity Amine sensor chip (Bruker) using PDEA after NHS/EDC activation resulting in 230-670 RU with Sierra SPR-32 system (Bruker). The chip was equilibrated with SPR running buffer (PBS 0.05% Tween pH 7.3). Upon binding, target analyte was accumulated on the surface increasing the refractive index. This change in the refractive index was measured in real time and plotted as response or resonance units versus time. The analyte gp64 protein was applied to the chip in serial dilutions with a flow rate of 30 μl/min. The association was performed for 120 seconds and the dissociation for 120 seconds. After each run, the chip surface was regenerated with 30 μl regeneration buffer (10 mM glycine pH 2.0) and equilibrated with running buffer. Binding studies were carried out by the use of the Sierra SPR-32 system (Bruker); data evaluation was operated via the Sierra Analyser software, provided by the manufacturer, by the use of the Langmuir 1:1 model (RI=0). Evaluated dissociation constants (K_{D}) were standardized against the immobilized protein and indicated. Shown is the change in refractive index measured in real time and plotted as response or resonance unit [RU] versus time [sec]. The results are shown in FIG. 3. The K_{D} for fusion protein 218433 was 53.9 nM (see FIG. 3A) and 31.1 nM for fusion protein 218441 (see FIG. 3B). The fusion proteins 220067 (SEQ ID NO: 8), 220068 (SEQ ID NO: 10), and 220069 (SEQ ID NO: 9) show similar K_{D} values less 60 nM for gp64.

The fusion protein comprises in addition to the gp64 binding protein (SEQ ID NO: 2 or SEQ ID NO: 3) at least one non-gp64 binding protein (SEQ ID NO: 6). SPR studies showed that SEQ ID NO: 6 or a dimer of SEQ ID NO: 6 or a protein with at least 89.5% identity to SEQ ID NO: 6 does not bind to gp64.

### Example 4. Affinity Purification

Coupling parameter. Fusion proteins comprising gp64 binding protein were purified to homogeneity and coupled for affinity chromatography (AIC) experiments. Purified fusion proteins comprising the gp64 binding protein were immobilized at 30 mg per mL activated Praesto™ Epoxy 85 (Purolite) according to the manufacturer's instructions, coupling conditions: 35° C. for 3 h, pH 9.5, 110 mg Na₂SO₄ per mL Resin. All gp64 ligands (fusion proteins) were successfully coupled to epoxy-activated Praesto 85 resin.

Elution profile. For elution pH determination, resins were packed into superformance column housing (Götec, 5-50) and equilibrated in 25 mM Tris, 0.02% (w/v) Tergitol 15-S-9 pH 8.0. Resin was loaded with 90-300 mL of the supernatant of the cultured insect host cells (Sf9-supernatant) containing gp64 with residence times of 1 or 4 minutes. Wash steps with up-flow were inserted after loading of 50 mL to avoid column clogging for loading of 300 mL supernatant. Elution was performed with a gradient from pH 6.0 to pH 2.0 in 20 column volumes (CV) using 100 mM citric acid 0.02% (w/v) Tergitol 15-S-9. The pH of buffer fractions containing the target was determined. Both fusion protein 218433 comprising binding protein SEQ ID NO: 2 and fusion protein and 218441 comprising binding protein SEQ ID NO: 3 showed a homogenous elution profile with a singular peak. For both fusion proteins, the peak maximum was at pH 3.2 in a pH gradient profile. Eluted gp64 showed high purity. The pH of eluted gp64 was adjusted to 8.0 (neutralized). The remaining binding affinity of purified gp64 to 218433 was confirmed by SPR. Furthermore, the eluted and neutralized gp64 was reloaded on Praesto 85_218433 or Praesto 85_218441; no gp64 was detected in flowthrough, and the gp64 bound to Praesto 85_218433 or Praesto 85_218441 was eluted with pH 3.2. Elution and neutralization does not influence ability of binding gp64 to 218433 or 218441.

Static binding capacity (SBC). The resin with immobilized 218433 or 218441 (Praesto 85_218433 or Praesto 85_218441) was equilibrated in 25 mM Tris, 0.02% (w/v) Tergitol 15-S-9 pH 8.0. Praesto 85_218433 or Praesto 85 218441 purified gp64 (1 mL, 0.6 mg/mL) was mixed with 20 μl of the resin for 1 h at RT. The matrix was washed 2 times with 300 μl with 25 mM Tris, 0.02% (w/v) Tergitol 15-S-9 pH 8.0 and the bound protein was eluted 100 μl with 100 mM citric acid 0.02% (w/v) Tergitol 15-S-9 pH 2.0. The static binding capacity (SBC) was determined by the mass eluted protein calculated by UV280 nm absorption and the extinction coefficient of gp64. The static binding capacity was 7.7 mg/ml for Praesto 85_218433 and 2 mg/ml for Praesto 85_218441.

Caustic stability. 218433 was coupled to Praesto Epoxy 85 as described above and treated with 0.1 M NaOH for 10 h at RT. The remaining SBC was 93% for 218433 (equals 40 CIP cycles).

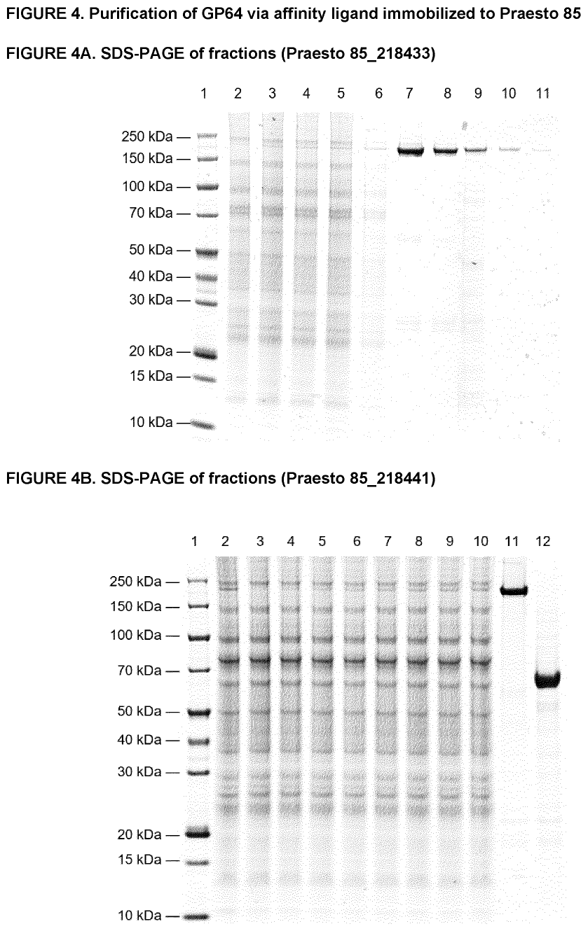

Characterization of gp64 after elution by SDS-PAGE. For purity analyses, 0.75 μg and 0.5 μg of neutralized gp64 from Praesto 85_218433 or Praesto 85_218441 elution fraction was used as sample. Samples were not reduced. Purity of eluted gp64 was analyzed by SDS-PAGE (NuPAGE system Invitrogen, 4-12% Bis-Tris-Gel. Staining: Coomassie Blue R250. Densitometric evaluation software: TotalLab 1D). Eluted gp64 from Praesto 85_218433 or Praesto 85_218441 had a purity of >90%. Results are shown in FIG. 4.

Characterization of gp64 after elution by binding analysis (SPR). Neutralized eluted gp64 from Praesto 85_218433 or Praesto 85_218441 was used as sample and analyzed for binding against 218433. 218433 was immobilized via Cysteines on High Capacity Amine SPR Affinity Sensor (Bruker) by PDEA reaction. In the experiment, the binding of eluted and neutralized gp64 to 218433 was confirmed.

Western blot. Fractions of AIC-runs via Praesto 85_218433 or Praesto 85_218441 were analyzed in Western Blot. Fractions were loaded and run on 4-15% Gel (4-15% Mini-PROTEAN TGX Stain-Free Gel) and transferred to PVDF membrane via Trans-Blot Turbo™ Transfer System (Biorad). For detection of gp64 AcmNPV gp64 polyclonal Antibody (Invitrogen) as primary antibody and Goat-Anti-Rabbit IgG-HRP as secondary antibody (Invitrogen) was used. Signals were visualized by HRP-ECL reaction.

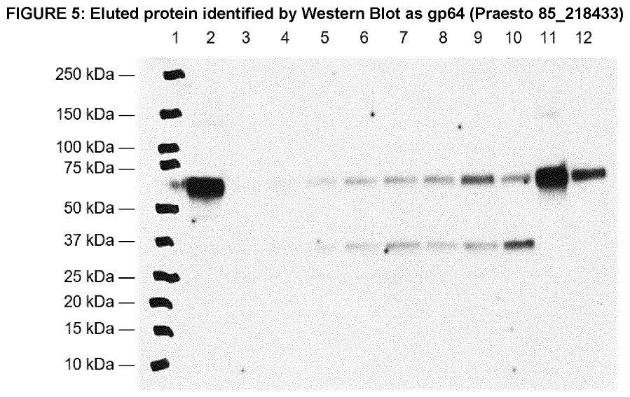

Eluted protein of AIC runs with Praesto 85_218433 or Praesto 85_218441 was identified as gp64 in Western Blot with gp64 specific antibody. The amount of gp64 in the flowthrough was considerably reduced compared to gp64 containing Sf9 supernatant. Results are shown in FIG. 5.

1. A baculoviral envelope protein gp64 binding protein comprising an amino acid sequence with at least 90% sequence identity to SEQ ID NO: 1, SEQ ID NO: 2, or SEQ ID NO: 3, wherein the baculoviral envelope protein qp64 binding protein has a binding affinity of less than 100 nM for gp64.

2. The baculoviral envelope protein qp64 binding protein according to claim 1, wherein 2, 3, 4, 5, or 6 binding proteins are linked to each other.

3. The baculoviral envelope protein qp64 binding protein according to claim 1, wherein the baculoviral envelope protein gp64 binding protein is fused to at least one non-gp64 binding protein.

4. The baculoviral envelope protein qp64 binding protein according to claim 1, wherein the baculoviral envelope protein ap64 binding protein is fused to one or two non-gp64 binding protein(s) with at least 89% identity to SEQ ID NO: 6.

5. A fusion protein comprising the baculoviral envelope protein gp64 binding protein according to claim 1 and one or two non-gp64 binding protein(s) with at least 89% identity to SEQ ID NO: 6.

6. The fusion protein according to claim 5, wherein said fusion protein comprises an attachment site for site-specific coupling of the fusion protein to a solid support.

7. (canceled)

8. An affinity separation matrix comprising the baculoviral envelope protein qp64 binding protein according to claim 1 or a fusion protein thereof with one or two non-gp64 binding protein(s) with at least 89% identity to SEQ ID NO: 6.

9. (canceled)

10. A method for affinity capturing of gp64, the method comprising:
(i) providing a liquid that contains a gp64;
(ii) providing an affinity separation matrix comprising at least one baculoviral envelope protein qp64 binding protein according to claim 1 or a fusion protein thereof with one or two non-qp64 binding protein(s) with at least 89% identity to SEQ ID NO: 6, wherein the at least one baculoviral envelope protein qp64 binding protein or the fusion protein thereof is coupled to said affinity separation matrix;
(iii) contacting said affinity separation matrix with the liquid under conditions that permit binding of the at least one baculoviral envelope protein qp64 binding protein or the fusion protein thereof to the qp64; and
(iv) eluting said gp64 from said affinity purification matrix.

11. (canceled)

12. A method for determining an amount of gp64 in a liquid sample, the method comprising:
(i) providing a liquid that contains gp64;
(ii) providing a baculoviral envelope protein qp64 binding protein according to claim 1 or a fusion protein thereof with one or two non-gp64 binding protein(s) with at least 89% identity to SEQ ID NO: 6;
(iii) contacting the liquid with the baculoviral envelope protein qp64 binding protein or the fusion protein thereof under conditions that permit binding of the baculoviral envelope protein ap64 binding protein or the fusion protein thereof to the gp64 to form a complex;
(iv) isolating the complex; and
(v) determining an amount of the gp64 in the complex, thereby determining an amount of qp64 in the liquid.

13. (canceled)

14. A method for isolating a vaccine protein, the method comprising:
(i) providing a sample that contains a vaccine protein expressed in a baculovirus-transduced insect cell expression system;
(ii) providing at least one baculoviral envelope protein qp64 binding protein according to claim 1 or a fusion protein thereof with one or two non-gp64 binding protein(s) that have at least 89% identity to SEQ ID NO: 6;
(iii) contacting said baculoviral envelope protein gp64 binding protein or the fusion protein thereof with the sample under conditions that permit binding of the baculoviral envelope protein qp64 binding protein or the fusion protein thereof to baculoviral envelope protein gp64 present in the sample, thereby forming a complex of the baculoviral envelope protein qp64 or the fusion protein with the baculoviral envelope protein gp64;
(iv) removing the complex of the baculoviral envelope protein gp64 bound to said baculoviral envelope protein gp64 binding protein or the fusion protein from said sample that contains the vaccine protein; and
(v) isolating the vaccine protein from the sample.

15. The method according to claim 14, wherein in step (ii), the binding protein or the fusion protein thereof is coupled to an affinity separation matrix.


SEQUENCE LISTING (10 sequences)

SEQ ID NO: 1 - protein, 56 aa, synthetic construct
  REGION 1..56: note = gp64 binding protein
  SITE 23: note = Xaa - Xaa may be selected from any amino acid, preferably H or Y
  SITE 26: note = Xaa - Xaa may be selected from any amino acid, preferably S or N
  SITE 27: note = Xaa - Xaa may be selected from any amino acid, preferably W or I
  SITE 30: note = Xaa - Xaa may be selected from any amino acid, preferably R or Q
  SITE 31: note = Xaa - Xaa may be selected from any amino acid, preferably W or Y
  SITE 33: note = Xaa - Xaa may be selected from any amino acid, preferably S or M
  SITE 34: note = Xaa - Xaa may be selected from any amino acid, preferably D or Y
  SITE 35: note = Xaa - Xaa may be selected from any amino acid, preferably D or R
  SITE 40: note = Xaa - Xaa may be selected from any amino acid, preferably K or L
  SITE 41: note = Xaa - Xaa may be selected from any amino acid, preferably E or Q
  SITE 42: note = Xaa - Xaa may be selected from any amino acid, preferably T or V
  SITE 44: note = Xaa - Xaa may be selected from any amino acid, preferably E or R
  SITE 45: note = Xaa - Xaa may be selected from any amino acid, preferably Q or W
  SITE 48: note = Xaa - Xaa may be selected from any amino acid, preferably E or W
  SITE 49: note = Xaa - Xaa may be selected from any amino acid, preferably L or Q
  SITE 52: note = Xaa - Xaa may be selected from any amino acid, preferably R or Q
AKFDEAQSAADSEILHLPNLTEXQRXXFRXXLXXXPSVSXXXLXXAQXXNDXQAPK

SEQ ID NO: 2 - protein, 56 aa, synthetic construct
  REGION 1..56: note = gp64 binding protein (433)
AKFDEAQSAADSEILHLPNLTEHQRSWFRRWLMDDPSVSKETLRQAQWLNDRQAPK

SEQ ID NO: 3 - protein, 56 aa, synthetic construct
  REGION 1..56: note = gp64 binding protein (441)
AKFDEAQSAADSEILHLPNLTEYQRNIFRQYLSYRPSVSLQVLEWAQEQNDQQAPK

SEQ ID NO: 4 - protein, 173 aa, synthetic construct
  REGION 1..173: note = fusion protein comprising SEQ ID NO: 2 (218433)
AKFDEAQSAADSEILHLPNLTEHQRSWFRRWLMDDPSVSKETLRQAQWLNDRQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 5 - protein, 173 aa, synthetic construct
  REGION 1..173: note = fusion protein comprising SEQ ID NO: 3 (218441)
AKFDEAQSAADSEILHLPNLTEYQRNIFRQYLSYRPSVSLQVLEWAQEQNDQQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 6 - protein, 56 aa, synthetic construct
  REGION 1..56: note = non- Ig binding protein
AQHDKIQQAADKEILHLPNLTEEQRNKFRQSLRDDPSVSAEILAEAKKLNDAQAPK

SEQ ID NO: 7 - protein, 512 aa, synthetic construct
  REGION 1..512: note = gp64 protein target
MVSAIVLYVLLAAAAHSAFAAEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 8 - protein, 173 aa, synthetic construct
  REGION 1..173: note = fusion protein comprising gp64 binding ligand
AKFDEAQSAADSEILHLPNLTEHQRSWFQRWLMDDPSVSKETLRQAQWLNDRQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 9 - protein, 173 aa, synthetic construct
  REGION 1..173: note = fusion protein comprising gp64 binding ligand
AKFDEAQSAADSEILHLPNLTEHQRSWFRQWLMDDPSVSKETLRQAQWLNDRQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 10 - protein, 173 aa, synthetic construct
  REGION 1..173: note = fusion protein comprising gp64 binding protein
AKFDEAQSAADSEILHLPNLTEHQRSWFIRWLMDDPSVSKETLRQAQWLNDRQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC